(19) United States
(12) Patent Application Publication
(10) Pub. No.: US 2021/0363095 A1
(43) Pub. Date: Nov. 25, 2021

(54) METHOD CAPABLE OF REALIZING PREPARATION AND IN-SITU SEPARATION OF OLIGOMERIC RICINOLEATE

(71) Applicants: Nankai University, Tianjin (CN); Tianjin Nankai University Castor Engineering S&T Co., Ltd., Tianjin (CN)

(72) Inventors: Feng YE, TIANJIN (CN); Liangnian HE, TIANJIN (CN); Hongru LI, TIANJIN (CN); Gang LI, TONGLIAO (CN); Fei YOU, TIANJIN (CN); Qingrui WANG, TIANJIN (CN); Song GAO, TIANJIN (CN); Xing HE, TIANJIN (CN); Xiaoying CUI, TIANJIN (CN)

(21) Appl. No.: 17/339,940

(22) Filed: Jun. 5, 2021

Related U.S. Application Data
(63) Continuation of application No. PCT/CN2020/114192, filed on Sep. 9, 2020.

(30) Foreign Application Priority Data
Feb. 27, 2020 (CN) 202010124194.1

(51) Int. Cl. C07C 69/732; C07C 67/08; B01J 31/02

(52) U.S. Cl. CPC C07C 69/732; C07C 67/08; B01J 2231/49; B01J 31/0298; B01J 31/0284; B01J 31/0285

(57) ABSTRACT

The disclosure is about a method capable of realizing the preparation and in-situ separation of the oligomeric ricinoleate, which uses the ricinoleic acid as raw material, and uses a protonic acid-type ionic liquid as a catalyst to cause the dehydration and esterification reactions between ricinoleic acid molecules. By continuously distilling out the generated water under a reduced pressure, the oligomeric ricinoleate with a polymerization degree of 2 to 10 is obtained. After the reaction, a method of washing with water or static stratification is selected to recover the catalyst according to the miscibility of the catalyst and reaction system. In his disclosure, renewable raw materials are used, the process is clean and pollution-free, and the operation is simple.

### CROSS-REFERENCE TO RELATED APPLICATIONS

This application is a continuation of International Patent Application No. PCT/CN2020/114192 with a filing date of Sep. 9, 2020, designating the United States, now pending, and further claims priority to Chinese Patent Application No. 202010124194.1 with a filing date of Feb. 27, 2020. The content of the aforementioned applications, including any intervening amendments thereto, are incorporated herein by reference.

### TECHNICAL FIELD

The disclosure relates to a method for preparing a biodegradable water-based metal cutting oil lubricant oligomeric ricinoleate, and belongs to the field of cutting oil preparation technologies.

### BACKGROUND

Cutting is carried out on a new surface every time in metal cutting. In addition to external friction, there is also intermolecular friction between a cutting tool and the metal during cutting. Thus the interface temperature in a cutting area can reach 600° C. to 800° C. Such high temperature and high pressure can reduce the strength and hardness of the cutting tool. Therefore, cutting oil is always needed for cooling, lubrication, cleaning, and rust prevention in metal processing.

At present, common cutting oils encounter the problems of low flash point, heavy smoke formation in high-speed cutting and thus renders the high danger coefficient in operation. Besides, the fast volatilization of cutting oils increases the invalid consumption and results in the high cost of the user. In addition, most cutting oils use mineral oil as base oil, which has insufficient oiliness and extreme pressure. To improve the properties of the cutting oils, excessive chlorides, sulfides are often added, which are detrimental to the environment as harmful substances can be generated during use and even after use.

The oligomeric ricinoleate, which is synthesized via the intermolecular esterification of ricinoleic acid, has characteristics of good biodegradability, easy emulsification, good dispersion and uniform film formation. It can be used as the lubricant in metal cutting liquid and is considered as a green chemical product with a great development prospect.

The traditional synthesis protocol of the oligomeric ricinoleate relay on the catalysis of protonic acids, such as sulfuric acid, p-toluenesulfonic acid etc. However, the homogeneous protonic acid catalysis has the problems of equipment corrosion, complicated post-treatment and even product color deepening which reduce the efficiency of the process and affect the product quality.

In order to avoid the above problems, the enzymatic method is proposed, referring to: (a) Bódalo-Santoyo, A.; Bastida-Rodríguez, J.; Máximo-Martín, M. F.; Montiel-Morte, M. C.; Murcia-Almagro, M. D. Enzymatic biosynthesis of ricinoleic acid estolides. Biochem. Eng. J. 2005, 26, 155-158. (b) Bódalo, A.; Bastida, J.; Máximo, M. F.; Montiel, M. C.; Gómez, M.; Murcia, M. D. Production of ricinoleic acid estolide with free and immobilized lipase from Candida rugosa. Biochem. Eng. J. 2008, 39, 450-456. (c) Bódalo, A.; Bastida, J.; Máximo, M. F.; Montiel, M. C.; Murcia, M. D.; Ortega, S. Influence of the operating conditions on lipase-catalysed synthesis of ricinoleic acid estolides in solvent-free systems. Biochem. Eng. J. 2009, 44, 214-219. (d) Horchani, H.; Bouaziz, A.; Gargouri, Y.; Sayari, A. Immobilized staphylococcus xylosus lipase-catalysed synthesis of ricinoleic acid esters. J. Mal. Catal. B: Enzym. 2012, 75, 35-42. (e) Yoshida, Y.; Kawase, M.; Yamaguchi, C.; Yamane, T. Syntheses of estolides with immobilized lipase. J. Jpn. Oil Chem. Soc. 1995, 44, 328-333. (f) Erhan, S. M.; Kleiman, R.; Isbell, T. A. Estolides from meadowfoam oil fatty acids and other monounsaturated fatty acids. J. Am. Oil Chem. Soc. 1993, 70, 461-465. Nevertheless, the enzymatic method has the disadvantage of high cost, low efficiency, and unstable operation, thus being not applicable for industrial production.

In recent years, the Lewis acid catalysts have been developed for the synthesis of oligomeric ricinoleate, such as stannous chloride, stannous octoate, and the like. These catalysts showed satisfying activity but encountered the problems of separation or metal ion residue. In contrast, the heterogeneous Lewis acid catalysts have characteristics of easy separation and no equipment corrosion, but the mass transfer between the phase interfaces can reduce a catalytic performance of these catalysts, referring to: (a) Haiyun Li, Yonglei Wang, Hongxia Fang, et al. synthesis and characterization of polyhydroxy polyricinoleic acid hyperdispersant. Applied chemical engineering, 2014, 43: 1064-1067. (b) Vadgama, R. N.; Odaneth, A. A.; Lali, A. M. New synthetic route for polyricinoleic acid with Tin (II) 2-ethylhexanoate. Heliyon 2019, 5, e01944. (c) Fei You, Hongru Li, Kaihong Chen, Fengge Zhao, Feng Ye, Xiaoying Cui, Liangnian He. Preparation method of oligomeric ricinoleate [P]: China, 201811438980.8. 2019.05.17.

The ionic liquid catalyst has both the characteristics of a high efficiency of a homogeneous catalyst and easy separation of a heterogeneous catalyst. Meanwhile, the structure and property of the ionic liquid catalyst can be designed, so that it is very attractive in organic synthesis. At present, the acidic ionic liquid catalyst [HSO₃-BMim]TS (1-butanesulfonic acid-3-methylimidazole p-toluene sulfonate) has been used in the synthesis of the oligomeric ricinoleate. However, its catalytic efficiency under the optimal operating condition is still far behind that of the Lewis acid catalysts such as stannous chloride and stannous octoate, thus failing to show a superiority of the ionic liquid as the catalyst. In addition, the relevant reports about the separation, recovery and reuse performances of the ionic liquid catalyst after reaction are still absent. Refer to: (a) Wang, G.; Sun, S. Synthesis of ricinoleic acid estolides by the esterification of ricinoleic acids using functional acid ionic liquids as catalysts. J. Oleo Sci. 2017, 66, 753-759.

Considering the designable features of the ionic liquid catalyst, the ionic liquid catalyst can be designed according to a catalytic mechanism of the esterification reaction, which will help to develop an efficient, green, and simply operated preparation process of the oligomeric ricinoleate.

### SUMMARY

The disclosure aims to provide a method for efficient preparation and separation of an oligomeric ricinoleate with the ionic liquid as a catalyst. According to the catalytic mechanism of the esterification reaction, the ionic liquids containing one or more ionizable protons are designed and synthesized as the catalyst for synthesis of the oligomeric ricinoleate. With these ionic liquid catalysts, the polymerization degree of the resulting oligomeric ricinoleate can be conveniently adjusted by the reaction time. As the oligomeric ricinoleate with different average polymerization degree has different application, this synthesis protocol has wide applications. For example, the oligomeric ricinoleate with an average polymerization degree of 4 can be added into metal cutting oil as a lubricant. The preparation process is simple, clean and pollution-free. After the reaction, the product oligomeric ricinoleate and the catalyst can be separated by washing with water or in-situ static stratification. More importantly, the catalyst may be recycled for many times, thus showing a commercial application prospect.

The technical solutions of the disclosure are as follows.

A method for preparation and in-situ separation of an oligomeric ricinoleate, which uses ricinoleic acid as the raw material and Bronsted acidic ionic liquid as the catalyst to cause the dehydration and esterification reactions between ricinoleic acid molecules under the reduced pressure, comprises the following specific preparation steps of:

step 1: putting the ricinoleic acid and the catalyst into a reaction flask;

step 2: starting the vacuum pump to adjust a vacuum degree of the reaction system, heating to a reaction temperature under a stirring condition, and starting the dehydration and esterification reactions;

step 3: after the reaction, removing the catalyst, and finally obtaining the oligomeric ricinoleate as product.

A reaction formula of the above preparation process is:

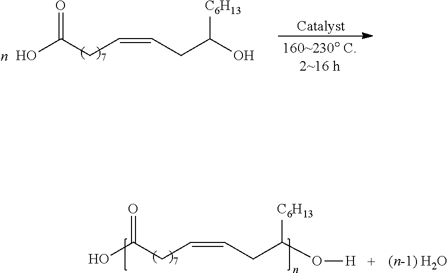

The catalyst is an ionic liquid catalyst, the cation of the ionic liquid catalysts contains one or more of N-methylpyrrolidone ion ([NMP]⁺), N-butylsulfonate pyridinium ion ([HSO_{-BPy]}⁺), 1-butancsulfonic, acid-3-methylimidazole ion ([HSO₃-BMim]⁺), N-(4-butanesulfonic aciditriethylamine ion ([HSO₃-BNEt₃]⁺), and 1-butanesulfonic acid-1,8-diazabicyclo[5.4.0]undec-7-ene ion ([HSO₃-BDBU]⁺). And the anion of the ionic liquid catalyst contains one or more of hydrogen sulfate (HSO₄⁻), dihydrogen phosphate (H₂PO₄⁻), tritluoromethanesulfonic acid ion (CF₃SO₄⁻), and p-toluenesulfonic acid ion (PTSA⁻), the ionic liquid catalyst can be the random combination of the above cations and anions, and in the reaction, the amount of the catalyst accounts for 1 wt. % to 30 wt. % of the total weight of the reaction system.

The method for separating the catalyst is washing with water or static stratification.

The raw material ricinoleic acid has an acid value of 150 mg KOH/g to 190 mg KOH/g

The dehydration and esterification reactions of ricinoleic acid is performed between 160° C. to 230° C. and under a vacuum degree of 70 kPa to 0 kPa, and last for 2 hours to 16 hours.

With the above mentioned conditions, the resulting oligomeric ricinoleate has an acid value of 20 mg KOH/g to 90 mg KOH/g, which means the polymerization degree is less than or equal to 10; at 40° C., a kinematic viscosity of the product is less than or equal to 1,000 mm²/s; and at 100° C., the kinematic viscosity is less than or equal to 100 mm²/s. A structural formula of the oligomeric ricinoleate is as follows:

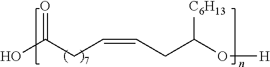

wherein, a is an integer from 2 to 10.

The disclosure has the advantages and positive effects as follows:

1. the raw material is the ricinoleic acid, which may be obtained by hydrolysis of castor oil with easy availability and low price. These materials is convenient for storage and transportation, thus having obvious advantages in industrial production;

2. the preparation process is simple and the production cycle is short and no side reactions occur;

3. the method avoids the use of solvent and needn't special equipment, which reduces the investment costs and saves energy consumption, and meanwhile, the whole process flow is environmentally friendly;

4. the method uses the ionic liquid catalyst, which may be recycled and reused for many times, thus greatly reducing the production costs, and having considerable economic benefits;

5. the oligomeric ricinoleate prepared by the disclosure has a good lubricating property and a low-temperature fluidity, which can be used as the cutting oil for metal processing, and belong to a clean and environmentally friendly product;

6. the yield of the oligomeric ricinoleate prepared by the method reaches more than 90%. Besides, the product are biodegradable, thus meeting requirements of environmental protection, and having an industrial production prospect.

### BRIEF DESCRIPTION OF THE DRAWINGS

FIG. 1 is the reaction formula for synthesis of oligomeric ricinoleate.

FIG. 2 is the FT-IR spectrum of a product.

FIG. 3 is the ¹H NMR spectrum of the product.

FIG. 4 is the ¹³C NMR spectrum of the product.

FIG. 5 is the ESI-MS spectrum of the product.

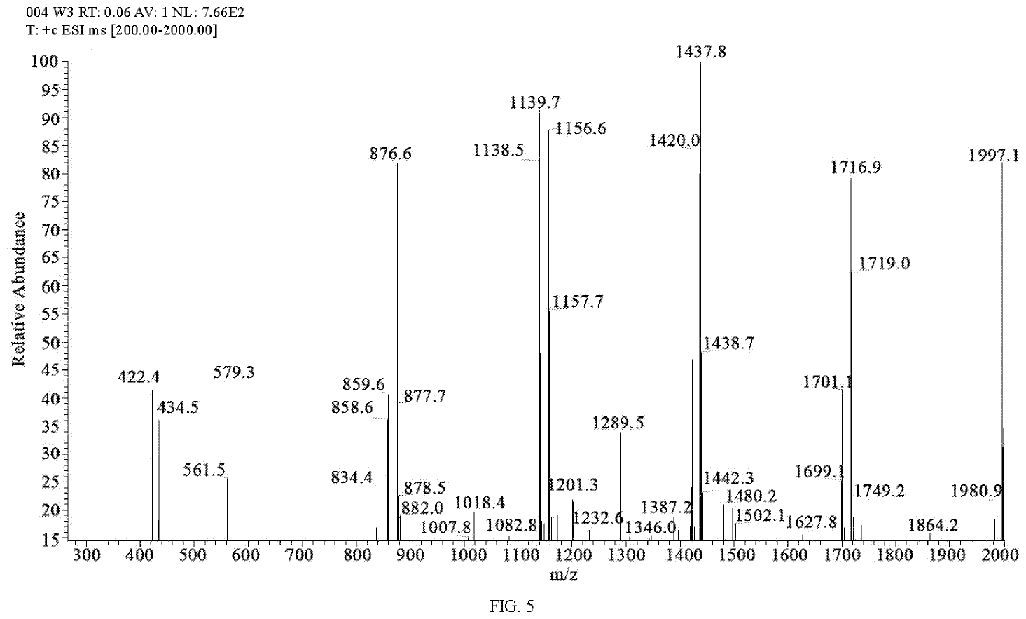

### DETAILED DESCRIPTION

The disclosure is about a method capable of realizing preparation and in-situ separation of the oligomeric ricinoleate. In order to meet needs of industrialization, an environmentally friendly and easily operated preparation method with high-yield of oligomeric ricinoleate is developed with the renewable ricinoleic acid as starting material through experimental screening. The positive effect of the disclosure contains the use of bio-base and renewable starting material, recyclable catalyst, simple operation, green process and adjustable product with excellent properties.

### Embodiment 1

10 g of ricinoleic acid and 0.5 g of N-methylpyrrolidone hydrosulfate ([NMP]HSO₄) were added into a reaction flask, a vacuum pump was started to adjust a vacuum degree of the reaction system to be 50 kPa, and meanwhile, the mixture was stirred and heated to 160° C. With the progress of the reaction, water was continuously pumped out, and the reaction was stopped 5 hours later. Then, the catalyst was removed by washing with water to obtain the product, with a yield of 93%. It was measured that the acid value of the product was 76 mg KOH/g, which corresponds to an average polymerization degree about 2. The kinematic viscosity of the resulting product was 163.8 mm² at 40° C., and the kinematic viscosity was 31.4 mm²/s at 100° C.

Preparation method of the catalyst [NMP]HSO₄: equivalent sulfuric acid was added into 0.1 mol (9.9 g) of N-methylpyrrolidone. The reaction mixture was stirred at 80° C. for 24 hours, and then dried in vacuum at 80° C. for 24 hours to obtain the [NMP]HSO₄.

### Embodiment 2

10 g of ricinoleic acid and 1 g of N-butylsulfonate pyridinium p-toluenesulfonate ([HSO₃-BPy]PTSA) were added into a reaction flask, a vacuum pump was started to adjust the vacuum degree of the reaction system to be 30 kPa, and meanwhile, the mixture was stirred and heated to 230° C. With the progress of the reaction, water was continuously pumped out, and the reaction was stopped 2 hours later. Then, the catalyst was removed by washing with water to obtain a product. The final yield of the product was 93% with an acid value of 27 mg KOH/g, which corresponded to an average polymerization degree about 8. The kinematic viscosity of the resulting product was 884.4 mm²/s at 40° C., and the kinematic viscosity was 89.7 mm²/s at 100° C.

Preparation method of the catalyst [HSO₃-BPy]PTSA: 0.05 mol (10.8 g) of N-butylsulfonate pyridinium inner salt was added into 50 mL of dichloromethane, and then equivalent p-toluenesulfonic acid was added. The reaction mixture was stirred at 60° C. for 4 hours, and then a viscous liquid on an upper layer was separated, which was then washed twice with ether, and dried in vacuum at 100° C. for 24 hours to obtain the [HSO₃-BPy]PTSA.

### Embodiment 3

10 g of ricinoleic acid and 1.5 g of N-(4-butanesulfonic acid) triethylamine dihydrogenphosphat ([HSO₃-BNEt₃]H₂PO₄) were added into a reaction flask, a vacuum pump was started to adjust the vacuum degree of the reaction system to be 70 kPa, and meanwhile, the mixture was stirred and heated to 200° C. With the progress of the reaction, water was continuously pumped out, and the reaction was stopped 6 hours later. Then, the catalyst was removed by washing with water to obtain the product. The final product yield was 92% with an acid value of 35 mg KOH/g, which corresponded to an average polymerization degree about 6. The kinematic viscosity of the resulting product was 712.5 mm²/s at 40° C., and the kinematic viscosity was 81.7 mm²/s at 100° C.

Preparation method of the catalyst [HSO₃-BNEt₃]H₂PO₄: 0.05 mol (11.9 g) of N-(4-butanesulfonic acid) triethylamine inner salt was added into 50 mL of dichloromethane, and then equivalent phosphoric acid was added. The reaction mixture was stirred at 60° C. for 4 hours, and then a viscous liquid on an upper layer was separated, which was then washed twice with ether, and dried in vacuum at 100° C. for 24 hours to obtain the [HSO₃-BNEt₃]H₂PO₄.

### Embodiment 4

10 g of ricinoleic acid and 2 g of 1-butanesulfonic acid-3-methylimidazolium trifluoromethanesulfonate ([HSO₃-BMim]CF₃SO₃) were added into a reaction flask, a vacuum pump was started to adjust the vacuum degree of the reaction system to be 30 kPa, and meanwhile, the mixture was stirred and heated to 210° C. With the progress of the reaction, water was continuously pumped out, and the reaction was stopped 4 hours later. Then, the catalyst was removed by washing with water to obtain the product. The final product yield was 94% with an acid value of 56 mg KOH/g, which corresponded to an average polymerization degree was about 3 to 4. The kinematic viscosity of the resulting product was 391.6 mm²/s at 40° C., and the kinematic viscosity was 42.2 mm²/s at 100° C.

Preparation method of the catalyst [HSO₃-BMim]CF₃SO₃: 0.05 mol (10.9 g) of 1-butanesulfonic acid-3-methylimidazolium inner salt was added into 50 mL of dichloromethane, and then equivalent trifluoromethanesulfonic acid was added. The reaction mixture was stirred at 60° C. for 4 hours, and then a viscous liquid on an upper layer was separated, which was then washed twice with ether, and dried in vacuum at 100° C. for 24 hours to obtain the [HSO₃-BMim]CF₃SO₃.

Embodiment 5

1 g of ricinoleic acid and 0.01 g of N-butylsulfonate pyridinium dihydrogenphosphat ([HSO₃-BPy]H₂PO₄) were added into a reaction flask, and reacted under a normal pressure, and meanwhile, the mixture was stirred and heated to 190° C. With the progress of the reaction, water was continuously pumped out, and the reaction was stopped 16 hours later. Then, the catalyst was removed by washing with water to obtain a product. The final product yield was 91% with an acid value of 20 mg KOH/g, which corresponded to an average polymerization degree about 9. The kinematic viscosity of the product was 911.4 mm²/s at 40° C., and the kinematic viscosity was 91.7 mm²/s at 100° C.

Preparation method of the catalyst [HSO₃-BPy]H₂PO₄: 0.05 mol (10.8 g) of N-butylsulfonate pyridinium inner salt was added into 50 mL of dichloromethane, and then equivalent phosphoric acid was added. The reaction mixture was stirred at 60° C. for 4 hours, and then a viscous liquid on an upper layer was separated, which was then washed twice with ether, and dried in vacuum at 100° C. for 24 hours to obtain the [HSO₃-BPy]H₂PO₄.

### Embodiment 6

0.5 g of ricinoleic acid and 0.025 g of N-(4-butanesulfonic acid) triethylamine disulfate ([HSO₃-BNEt₃]HSO₄) were added into a reaction flask, a vacuum pump was started to adjust a vacuum degree of a reaction system to be 70 kPa, and meanwhile, the mixture was stirred and heated to 210° C. With the progress of the reaction, water was continuously pumped out, and the reaction was stopped 3 hours later. Then, the catalyst was removed by washing with water to obtain a product. The final product yield was 93% with an acid value of 39 mg KOH/g, which corresponded to an average polymerization degree about 6. The kinematic viscosity of the product was 698.5 mm²/s at 40° C., and the kinematic viscosity was 79.5 mm²/s at 100° C.

Preparation method of the catalyst [HSO₃-BNEt₃]HSO₄: 0.05 mol (11.9 g) of N-(4-butanesulfonic acid) triethylamine inner salt was added into 50 mL of dichloromethane, and then equivalent sulfuric acid was added. The reaction mixture was stirred at 60° C. for 4 hours, and then a viscous liquid on an upper layer was separated, which was then washed twice with ether, and dried in vacuum at 100° C. for 24 hours to obtain the [HSO₃-BNEt₃] HSO₄.

### Embodiment 7

50 g of ricinoleic acid and 5 g of catalyst 1-butanesulfonic acid-1, 8-diazabicyclo [5 .4.0]undec-7- ene dihydrogenphosphat ([HSO₃-BDBU]H₂PO₄) were added into a reaction flask, a vacuum pump was started to control the vacuum degree of the reaction system to 50 kPa, and meanwhile, the mixture was stirred and heated to 170° C. With the progress of the reaction, water was continuously pumped out, and the reaction was stopped 16 hours later. When the reaction system was cooled to room temperature and standed for 1 hour, it could be found that the product and the catalyst were stratified with the product locating on the upper layer and the catalyst locating on the lower layer. Thus the product and the catalyst could be separated by simple dumping. The final yield of the product was 95% with an acid value of 17 mg KOH/g, which corresponded to an average polymerization degree about 10. The kinematic viscosity of the resulting product was 962.5 mm²/s at 40° C., and the kinematic viscosity was 95.0 mm²/s at 100° C.

Preparation method of the catalyst [HSO₃-BDBU]H₂PO₄: 0.05 mol (14.4 g) of 1-butanesulfonic acid-1,8-diazabicyclo[5.4.0]undec-7-ene inner salt was added into 50 mL of dichloromethane, and then equivalent phosphoric acid was added. The reaction mixture was stirred at 60° C. for 4 hours, and then a viscous liquid on an upper layer was separated, which was then washed twice with ether, and dried in vacuum at 100° C. for 24 hours to obtain the [HSO₃-BDBU]H₂PO₄.

### Embodiment 8 Investigation on Dosage of Catalyst

10 g of ricinoleic acid was added into a reaction flask, the usage of the catalyst 1 -butane sulfonic acid-1, 8-diazabicyclo[5.4.0]undec-7-ene dihydrogenphosphat ([HSO₃-BDBL]H₂PO₄) were set at respectively 1%, 5%, 15%, 20%, and 30% of a mass ratio to the raw material and a vacuum pump was started to regulate the vacuum degree of the reaction system to 50 kPa. Meanwhile, the mixture was stirred and heated to 200° C. With the progress of the reaction, water was continuously pumped out, and the reaction was stopped 5 hours later. When the reaction system was cooled to room temperature and standed for 1 hour, the product and the catalyst were separated by static stratification and simple dumping. The effect of the dosage of the catalyst on a yield and a property of the product could be obtained, as shown in Table 1.

TABLE 1
Physicochemical properties of products prepared with different
dosages of catalyst

| Dosage | | Average | | Kinematic viscosity | |
|---|---|---|---|---|---|
| of catalyst | Acid value | polymerization | | mm²/s | |
| wt. % | mg KOH/g | degree | Yield % | 40° C. | 100° C. |
| 1 | 120 | 1-2 | 91 | 120.3 | 21.4 |
| 5 | 103 | 1-2 | 91 | 147.8 | 27.7 |
| 15 | 52 | 4 | 93 | 406.6 | 45.3 |
| 20 | 50 | 4 | 92 | 420.5 | 48.3 |
| 30 | 40 | 5 | 94 | 710.5 | 80.2 |

### Embodiment 9 Investigation on Vacuum Degree of Reaction

10 g of ricinoleic acid and 1.5 g of catalyst 1-butanesulfonic acid-1, 8-diazabicyclo [5.4.0]undec-7- ene dihydrogenphosphat ([HSO₃-BDBU]H₂PO₄) were added into a reaction flask, a vacuum pump was started to adjust the vacuum degree of the reaction system between 0 kPa and 70 kPa respectively, and meanwhile, the mixture was stirred and heated to 190° C. With the progress of the reaction, water was continuously pumped out, and the reaction was stopped 8 hours later. When a reaction system was cooled to room temperature and standed for 1 hour, the product and catalyst were separated by static stratification and simple dumping. The effect of a vacuum degree on the yield and property of the product could be obtained, as shown in Table 2.

TABLE 2
Physicochemical properties of products prepared at different
vaccum degrees

| | | Average | | Kinematic viscosity | |
|---|---|---|---|---|---|
| Vacuum | Acid value | polymerization | | mm²/s | |
| degree kPa | mg KOH/g | degree | Yield % | 40° C. | 100° C. |
| 70 | 43 | 5 | 95 | 510.3 | 54.3 |
| 50 | 44 | 4-5 | 95 | 507.5 | 53.1 |
| 30 | 52 | 4 | 92 | 406.2 | 45.7 |
| 20 | 63 | 3 | 93 | 368.9 | 41.5 |
| 0 | 101 | 1-2 | 90 | 223.1 | 34.5 |

### Embodiment 10 Investigation on Recycling and Reuse of Catalyst

10 g of ricinoleic acid and 3 g of 1-butanesulfonic acid-1, 8-diazabicyclo [5.4.0 ]undec-7-ene dihydrogenphosphat ([HSO₃-BDBU]H₂PO₄) were added into a reaction flask, a vacuum pump was started to adjust the vacuum degree of the reaction system to be 50 kPa, and meanwhile, the mixture was stirred and heated to 220° C. With the progress of the reaction, water was continuously pumped out, and the reaction was stopped 5 hours later. Then, static stratification was carried out and the product A located on the upper layer was dumped and weighed to calculate the yield. The catalyst left in the reaction flask was weighed to be 2.8 g. 10 g of fresh ricinoleic acid was then added into the reaction flask containing the recycled catalyst to start another preparation process for oligomeric ricinoleate. With the same procedure, the above esterification operation was lasted for 5 hours, and then the product and the catalyst were separated by static stratification and dumping again to obtain the product B. Meanwhile, the remaining catalyst was weighed as 2.7 g, and 10 g of fresh ricinoleic acid was continuously added into the reaction flask containing the recycled catalyst. With the same operating conditions, the reaction was lasted for 5 hours to obtain the product C. Results of physicochemical properties of the products A, B and C measured refer to Table 3.

TABLE 3
Physicochemical properties of products prepared by recycling catalyst

| | | Average | | Kinematic viscosity | |
|---|---|---|---|---|---|
| | Acid value | polymerization | | mm²/s | |
| Product | mg KOH/g | degree | Yield % | 40° C. | 100° C. |
| A | 44 | 5 | 93 | 568.6 | 64.8 |
| B | 47 | 4-5 | 94 | 519.4 | 59.4 |
| C | 52 | 4 | 94 | 493.5 | 57.2 |

The embodiment indicates that the ionic liquid has a good stability and can be reused in synthesis of an oligomeric ricinoleate.

### Embodiment 11 Effect of Combination of Catalysts on Reaction

10 g of ricinoleic acid and 2 g of mixture of 1-butanesulfonic acid-3-methylimidazolium trifluoromethanesulfonate ([HSO₃-BMim]CF₃SO₃) and N-butylsulfonate pyridinium dihydrogenphosphat ([HSO₃-BPy]H₂PO₄) with a mass ratio of 1:1 were added into a reaction flask, a vacuum pump was started to adjust the vacuum degree to be 50 kPa, and meanwhile, the mixture was stirred and heated to 230° C. With the progress of the reaction, water was continuously pumped out, and the reaction was stopped 8 hours later. The catalyst was removed by washing with water. The final yield of the product was 92% with an acid value of the product to be 20 mg KOH/g, which corresponded to an average polymerization degree about 9. The kinematic viscosity of the product was 920.1 mm²/s at 40° C., and the kinematic viscosity was 92.3 mm²/s at 100° C.

Characterization of product oligomeric ricinoleate:

Appearance: yellow oily liquid

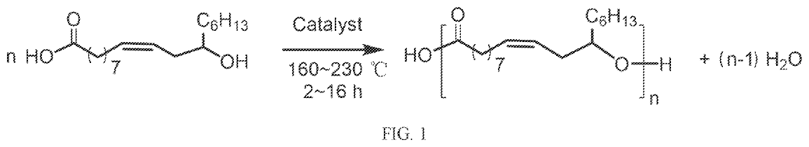

FT-IR (KBr) Vmax/cm⁻¹: 3416.44, 3010.55, 2927.89, 2855.81, 1733.38, 1711.66, 1464.22, 1245.41, 1183.74, 725.11; ESI-MS: m/z (+) 579.3, 876.6, 1139.7, 1437.8, 1716.9, 1997.1 (as shown in FIG. 1).

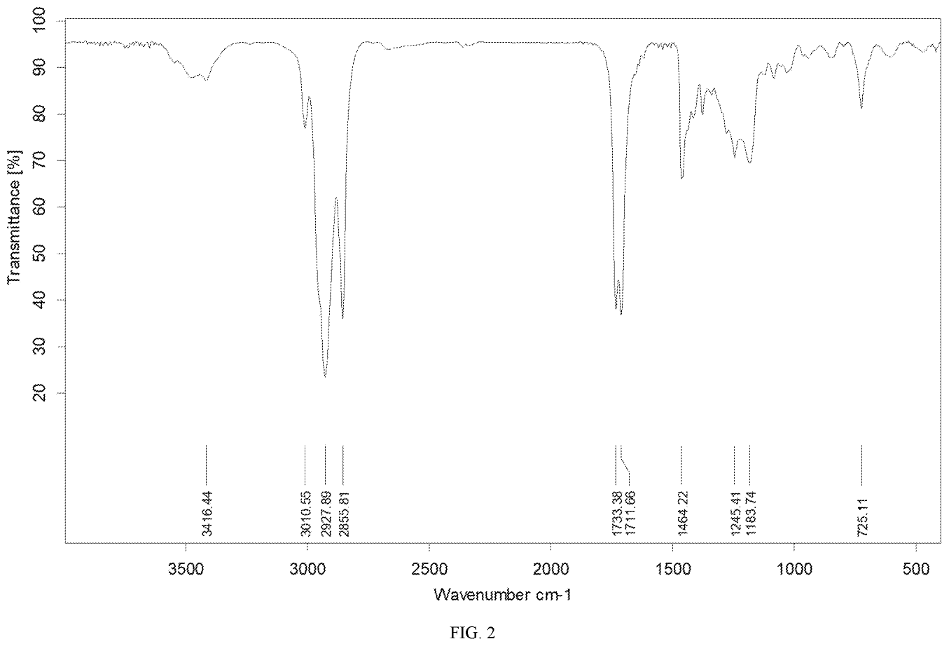

¹H NMR (400 MHz, CDCl₃) δ: 0.85-0.88 (t, J=3.9 Hz, 3H), 1.26-1.29 (m, 16H), 1.51-1.60 (m, 4H), 2.00-2.01 (m, 2H), 2.25-2.26 (m, 4H), 4.86-4.89 (m, 1H), 5.30-5.46 (m, 2H) ppm (as shown in FIG. 2).

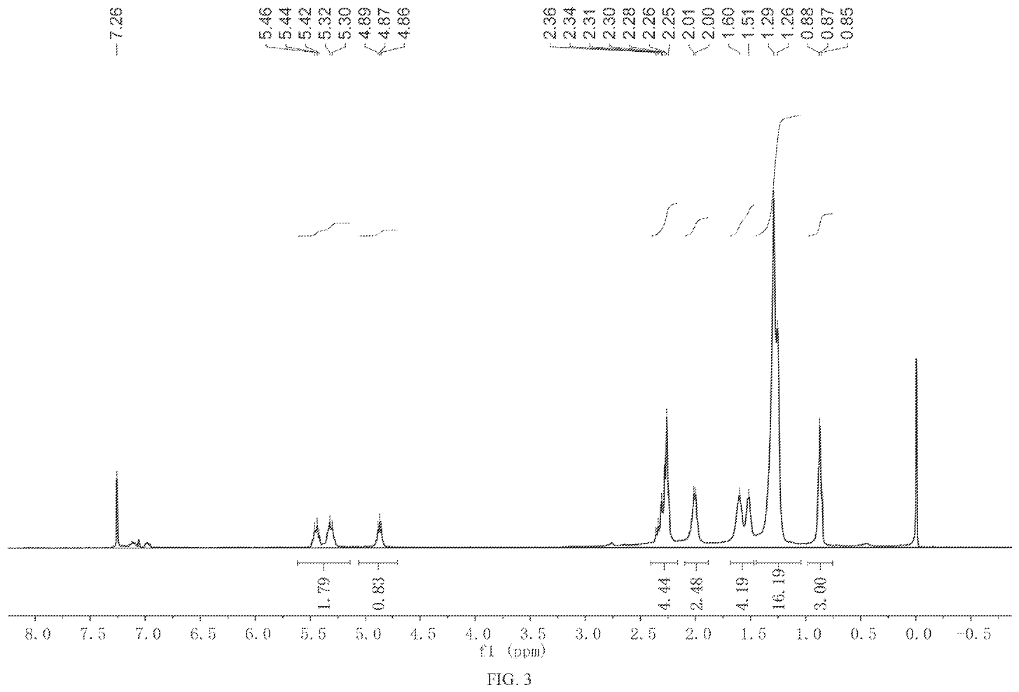

¹³C NMR (100 MHz, CDCl₃) δ: 14.08, 22.58, 25.11, 25.35, 27.20-27.35, 29.03-29.71, 31.75, 31.98, 33.62, 34.65, 73.69, 124.30, 132.51, 173.58 ppm (as shown in FIG. 3).

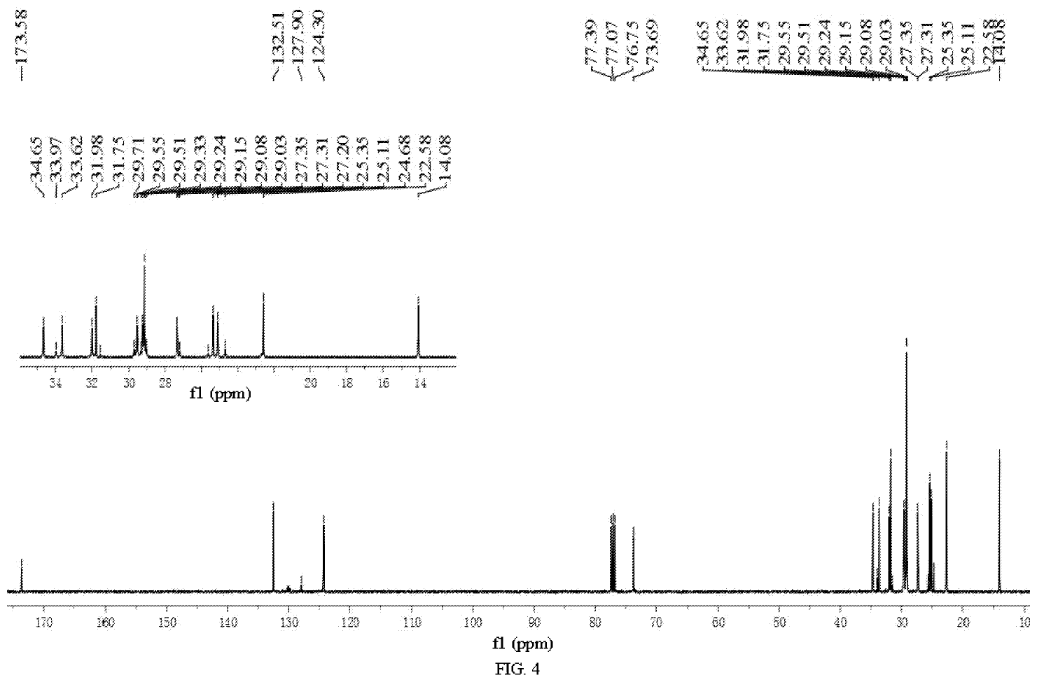

ESI-MS: m/z (+) 579.3, 876.6, 1139.7, 1437.8, 1716.9, 1997.1 (as shown in FIG. 4).

The foregoing describes several embodiments of the disclosure in detail, which cannot be regarded as limiting the implementation scope of the disclosure. All equal changes and improvements made according to the application scope of the disclosure shall still fall within the coverage scope of the patent of the disclosure.

What is claimed is:

1. A method capable of realizing preparation and in-situ separation of the oligomeric ricinoleate, which uses the ricinoleic acid as raw material, and uses a Bronsted acidic ionic liquid as catalyst to cause dehydration and esterification reactions between ricinoleic acid molecules, and takes out generated water by using a vacuum pump, thus being capable of obtaining an oligomeric ricinoleate with a polymerization degree lower than 10, comprising the following steps of:
step 1: putting the ricinoleic acid and the catalyst into a reaction flask;
step 2: starting the vacuum pump to adjust the vacuum degree of the reaction system, heating to a reaction temperature under a stirring condition, and starting the dehydration and esterification reactions;
step 3: after the reaction, removing the catalyst, and finally obtaining a product, which is the oligomeric ricinoleate.

2. The method capable of realizing the preparation and in-situ separation of the oligomeric ricinoleate according to claim 1, wherein after the reaction, the catalyst is removed by a method of washing with water or static stratification according to miscibility of the ionic liquid catalyst with the reaction system; for the ionic liquid that is soluble in the reaction system at room temperature, water washing was used to separate the catalyst with the product while for the ionic liquid which is immiscible with the reaction system at room temperature, static stratification and dumpling was applied.

3. The method capable of realizing the preparation and the in-situ separation of the oligomeric ricinoleate according to claim 1, wherein the used raw material ricinoleic acid has an acid value of 150 mg KOH/g to 190 mg KOH/g.

4. The method capable of realizing the preparation and the in-situ separation of the oligomeric ricinoleate according to claim 1, wherein the cation of the ionic liquid catalyst is one or more of N-methylpyrrolidone ion ([NMP]⁺), N-butylsulfonate pyridinium ion ([HSO₃-BPy]⁺), N-(4-butanesulfonic acid)triethylamine ion ([HSO₃-BNEt₃]⁺), 1-butanesulfonic acid-3-methylimidazole ion ([HSO₃-BMim]⁺), or 1-butanesulfonic acid-1,8-diazabicyclo[5.4.0]undec-7-ene ion ([HSO₃-BDBU])⁺); and the anion of the ionic liquid catalyst is one or more of hydrogen sulfate (HSO₄⁻), dihydrogen phosphate (H₂PO₄⁻), trifluoromethanesulfonic acid ion (CF₃SO₃⁻), or p-toluenesulfonic acid ion PTSA⁻).

5. The method capable of realizing the preparation and the in-situ separation of the oligomeric ricinoleate according to claim I wherein a dosage of the catalyst ranges from 1 wt. % to 30 wt. %.

6. The method capable of realizing the preparation and the in-sitz separation of the oligomeric ricinoleate according to claim 1, wherein the dehydration and esterification reactions are performed at the temperature of 160° C. to 230° C. and a vacuum degree of 70 kPa to 0 kPa, and last for 2 hours to 16 hours.

7. The method capable of realizing the preparation and the in-situ separation of the oligomeric ricinoleate according to claim 1, wherein the obtained oligomeric ricinoleate has an acid value of 10 mg KOH/g to 90 mg KOH/g, which corresponds to a polymerization degree less than or equal to 10; at 40° C., the kinematic viscosity of the product is less than or equal to 1,000 mm²/s; and at 100° C. the kinematic viscosity is less than or equal to 100 mm²/s.

8. An oligomeric ricinoleate prepared by the method of claim 1, wherein the final product oligomeric ricinoleate has a structural formula as follows: 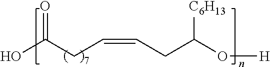
wherein, n is an integer from 2 to 10.

9. The oligomeric ricinoleate according to claim 8, wherein after the reaction, the catalyst is removed by a method of washing with water or static stratification according to miscibility of the ionic liquid catalyst with the reaction system; for the ionic liquid that is soluble in the reaction system at room temperature, water washing was used to separate the catalyst with the product while for the ionic liquid which is immiscible with the reaction system at room temperature, static stratification and dumpling was applied.

10. The oligomeric ricinoleate according to claim 8, wherein the used raw material ricinoleic acid has an acid value of 150 mg KOH/g to 190 mg KOH/g.

11. The oligomeric ricinoleate according to claim 8, wherein the cation of the ionic liquid catalyst is one or more of N-methylpyrrolidone ion ([NMP ]⁺), N-butylsulfonate pyridinium ion ([HSS₃-BPyr]⁺), N-(4-butanesulfonic acid)triethylamine ion ([HSO₃-BNEt₃]⁺), 1-butanesulfonic acid-3-methylimidazole ion ([HSO₃-BMim]⁺), or 1-butanesulfonic acid-1,8-diazabicyclo[5.4.0]undec-7-ene ion ([HSO₃-BDBU])⁺); and the anion of the ionic liquid catalyst is one or more of hydrogen sulfate (HSO₄⁻), dihydrogen phosphate (H₂PO₄⁻), trifluoromethanesulfonic acid ion (CF₃SO₃⁻), or p-toluenesulfonic acid ion PTSA⁻).

12. The oligomeric ricinoleate according to claim 8, wherein a dosage of the catalyst ranges from 1 wt. % to 30 wt. %.

13. The oligomeric ricinoleate according to claim 8, wherein the dehydration and esterification reactions are performed at the temperature of 160° C. to 230° C. and a vacuum degree of 70 kPa to 0 kPa, and last for 2 hours to 16 hours.

14. The oligomeric ricinoleate according to claim 8, wherein the obtained oligomeric ricinoleate has an acid value of 10 mg KOH/g to 90 mg KOH/g, which corresponds to a polymerization degree less than or equal to 10; at 40° C., the kinematic viscosity of the product is less than or equal to 1,000 mm²/s; and at 100° C., the kinematic viscosity is less than or equal to 100 mm²/s.
